(19) United States
(12) Patent Application Publication
(10) Pub. No.: US 2010/0094140 A1
(43) Pub. Date: Apr. 15, 2010

(54) Noninvasive Method and Apparatus to Measure Body Pressure Using Extrinsic Perturbation

(76) Inventors: Mindaugas Pranevicius, New York, NY (US); Osvaldas Pranevicius, New York, NY (US)

Correspondence Address: MINDAUGAS PRANEVICIUS, 111-37 75TH RD, FOREST HILLS, NY, 11375, US

(21) Appl. No.: 12/034,010

(22) Filed: Feb. 20, 2008

Related U.S. Application Data
(60) Provisional application No. 60/973,768, filed on Sep. 20, 2007.

(51) Int. Cl. A61B 5/02

(52) U.S. Cl. 600/485; 600/490; 600/493; 600/494; 600/495

(57) ABSTRACT

Current noninvasive blood pressure measurement methods are not able to measure pressure during nonpulsatile blood flow. We propose method to measure intravascular or other compartment pressure which applies extrinsic pressure oscillation. Pressure-volume response of the compressed structure is obtained and compartment pressure is estimated as the extrinsic pressure at which compressed structure has the highest compliance. Delivering extrinsic oscillations at a higher frequency than the pulse rate, pressure reading can be obtained much faster. Because it is not dependant on intrinsic vascular oscillations, pressure can be measured during arrhythmias, during cardiac bypass, during resuscitation, in the venous compartment or in the other nonpulsatile compressible body compartments.

### BACKGROUND

1. Field of Invention

This invention relates to a noninvasive blood pressure measurement method and devices in particular where pressure is measured using oscillatory method. An improved measurement method extends the application field and allows to measure pressure in other compressible body compartments (venous, ocular, bladder, intraabdominal, intrathoracic).

2. Prior Art

Noninvasive blood pressure is routinely measured in health and disease and is most important vital sign in assessing systemic perfusion. Historically palpation of the pulse was used to estimate blood pressure. Later the direct measurements were performed in horses (Hales, 1733) and during limb amputation. Direct measurement is invasive, requires placement of arterial catheter and is not practical for the routine use. Complications of the invasive monitoring include damage to the artery and surrounding structures, thrombosis, infection, bleeding, emboli, and unintended medication injection. Limb loss and stroke was described after invasive monitoring. Noninvasive measurement methods evolved subsequently.

### Noninvasive Blood Pressure Measurement Methods

Noninvasive blood pressure measurement methods are based on palpation, oscillometry, auscultation, tonometry (U.S. Pat. No. 3,926,179, U.S. Pat. No. 4,269,193, U.S. Pat. No. 6,514,211, U.S. Pat. No. 7,052,465), pletysmography (U.S. Pat. No. 5,447,161, U.S. Pat. No. 5,423,322), Doppler detection of flow or arterial wall motion or combination of these methods (U.S. Pat. No. 5,626,141, U.S. Pat. No. 6,045,509). Riva-Rocci cuff is commonly used to provide controlled compression of arterial wall. Automatic noninvasive blood pressure (NIBP) monitors commonly employ oscillometric method (U.S. Pat. No. 7,014,611, U.S. Pat. No. 5,255,686). Blood pressure cuff is inflated to occlude the artery and then pressure in the cuff is slowly released. Appearance of the pressure oscillations in the cuff is caused by arterial wall pulsation and peak oscillations occur when the cuff pressure approaches mean arterial pressure. Measurement algorithms are optimized to match sphygmomanometer readings.

### Reliance of Noninvasive Measurement Methods on Arterial Pulsation and Related Limitations

All noninvasive blood pressure monitors detect pulsatile blood flow related parameter (palpated pulse, Doppler, Korotkoff sounds, oscillations in the pressure, blood volume, blood flow or arterial wall). In other words all these methods “passively” register intrinsic oscillations in the arterial system and how they change when extrinsic pressure is applied. These methods are not applicable and do not work when the flow is nonpulsatile or arterial pulsations are diminished (shock, arrest, cardiac bypass, assist cardiac devices).

Sampling over the several cardiac cycles to register intrinsic oscillations is mandatory, what prolongs measurement (pressure in the cuff has to be changed slowly to obtain accurate reading). Prolonged measurements interfere with intravenous infusions pulse oxymetry monitoring in the same extremity. Repetitive prolonged and frequent measurements can cause extremity swelling, compartment syndrome and nerve injury.

Noninvasive pressure monitors based on volume clamp method or applanation tonometry apply variable pressure to compensate for intravascular pressure and volume changes (U.S. Pat. No. 4,807,638, U.S. Pat. No. 5,255,686). These gives advantage of monitoring the pressure continuously, however they are prone to errors in clinical setting, require sensor placement directly over the artery and still depend upon intravascular pressure oscillations. Intrinsic oscillations are not identical due to variable stroke volume and pulse pressure, what makes measurement over few cardiac cycles inaccurate. To address this extrinsic oscillation to calibrate the signal was proposed by Caro, U.S. Pat. No. 6,045,509. Measurement still depends on intrinsic pressure oscillations. None of the noninvasive methods can measure blood pressure during circulatory standstill, nonpulsatile blood flow. Measurements become inaccurate with irregular rhythm. Extremely high or low pressure measurements are also unreliable. Due to inability to measure blood pressure in these situations with current noninvasive blood pressure monitors one has to use invasive technique with all the associated risks and limitations.

Situations where current NIBP measurements are inadequate are not uncommon in the general population, but even more frequent in the critically ill patients. Current NIBP measurements become inadequate when the circulatory status changes rapidly (trauma, transport, military evacuation, arrest requiring ACLS, shock, surgery, etc.). Having blood pressure reading in these patients during critical period of hemodynamic instability is vital for decision making ant currently mandates invasive monitoring. If arterial line is not in place before the hemodynamic instability occurs (most common situation as few patients have arterial line placed preemptively), it may be difficult to place due to the weak pulse. Placing arterial line also requires specialized equipment and skilled, highly trained personnel. Arterial line is not placed in the field, where most critical patients present. In these situations manual measurement using auscultation is commonly unreliable. Palpation of pulse again has its limitations in these situations. Even though it is time proved method practiced for hundreds of years, it is subjective, notoriously inaccurate, provides qualitative rather than quantitative assessment and is operator skill dependant.

In these critical life threatening situations with absent or diminished arterial pulse simple and reliable method to measure arterial pressure noninvasively is desirable.

### OBJECTS AND ADVANTAGES

The purpose of our invention is noninvasive blood pressure measurement method which would not require intrinsic blood pressure oscillation and could also measure pressure in the other body compartments noninvasively. In addition to that it:
Would not require invasive arterial line placement;
Would measure noninvasively;
Would be easy to perform and would not depend on the operator skills;
Would allow measurements not only in the hospital setting with invasive monitoring capabilities but in any situation including ambulatory, field, transport or home setting;
Could measure extremely low blood pressure;
Could measure extremely high blood pressure;
Could measure rapidly changing blood pressure;
Could measure blood pressure during shock, when arterial pulsation is diminished;
Could measure blood pressure noninvasively during nonpulsatile flow (cardiac bypass, cardiac assist device);
Could measure blood pressure during arrest to assess effectiveness of CPR during resuscitation;
Could measure blood pressure during arrhythmias;
Would provide accurate measurements for patients with diminished pulsatility of arterial wall;
Could measure venous pressure;
Could measure ocular pressure;
Could measure bladder pressure;
Could measure abdominal pressure;
Could measure any other compressible body compartment pressure.

### DRAWINGS—FIGURES

FIG. 1 shows noninvasive blood pressure measurement device 10 connected to a pressure cuff 20.

FIG. 2A shows blood pressure volume V dependence on transmural pressure Pa−Pe. Compliance C=dV/dP is shown in the same graph, whereas maximal compliance reaches maximum Cmax, when arterial pressure equals external pressure Pa=Pe.

FIG. 2B shows blood volume oscillations dV (normalized) with gradual compression of the artery by external pressure Pe, whereas maximal oscillations occur when Pe=Pa.

FIG. 3 shows blood pressure measurement algorithm using extrinsic oscillation, where blood pressure equals to external compression pressure Pe with maximal compliance Cmax.

FIG. 4 shows blood pressure measurement algorithm when the plurality of compliance maximums is obtained during the measurement of pulsatile or variable blood pressure and minimum, maximum and mean values are displayed.

FIG. 5A shows that maximal volume oscillations occur when cuff pressure Pe intersects pulsatile arterial pressure Pa.

FIG. 5B shows that when arterial pulsations are diminished no clear volume oscillations are registered even when cuff pressure equal arterial pressure (Pe=Pa).

FIG. 6A shows that nonpulsatile arterial pressure can be measured using extrinsic perturbation. Maximal induced arterial volume oscillation is registered when Pe=Pa.

FIG. 6B shows that maximal calculated compliance is found when Pa=Pe.

FIG. 7A shows superimposed induced (extrinsic) and arterial pulse related (intrinsic) oscillations.

FIG. 7B shows the plurality of compliance maximums when arterial pressure fluctuates between maximal (systolic) and minimal (diastolic) values.

### REFERENCE NUMERALS

Pe External (cuff) pressure

Pa Arterial pressure

V Blood volume under the cuff

Posc Extrinsic pressure Pe oscillation

Vosc induced blood volume V oscillation

C Compliance, C=−Vosc/Posc

Cmax Maximal compliance (when Pe=Pa)

10 Blood pressure measurement apparatus

20 Inflatable pressure cuff

30 Blood vessel with blood volume V

40 Oscillator for repetitive cuff pressure perturbation Posc

50 Manometer (pressure sensor) for sensing cuff pressure (Pe)

60 Blood volume V sensor (plethysmograph)

70 CPU for data acquisition, control, processing, compliance C calculation display and user control execution

80 Display

90 Body portion containing the blood vessel

100 Cuff connecting hose

110 Pump and valves for cuff pressure control

120 User controls

### SUMMARY

We propose a method and apparatus to measure blood pressure in the vessel noninvasively irrespective whether the vessel has or does not have pulsatile flow or pressure. This method overcomes the reliance of noninvasive blood pressure measurements on the pulsatile flow. Method introduces extrinsic perturbation to the vascular bed, while extrinsic pressure is being changed and measures the response. This blood pressure measurement method does not require obtaining oscillations throughout multiple cardiac cycles nor does it require intrinsic oscillations to be of the same magnitude. Rather it delivers extrinsic oscillations, that can be delivered at a higher than the heart rate. That allows completing measurement faster. As the reliance on intrinsic oscillation is eliminated, the method can be applied to measure pressure in other nonpulsatile compressible body compartments (venous/abdominal/bladder/ocular etc.).

### DETAILED DESCRIPTION

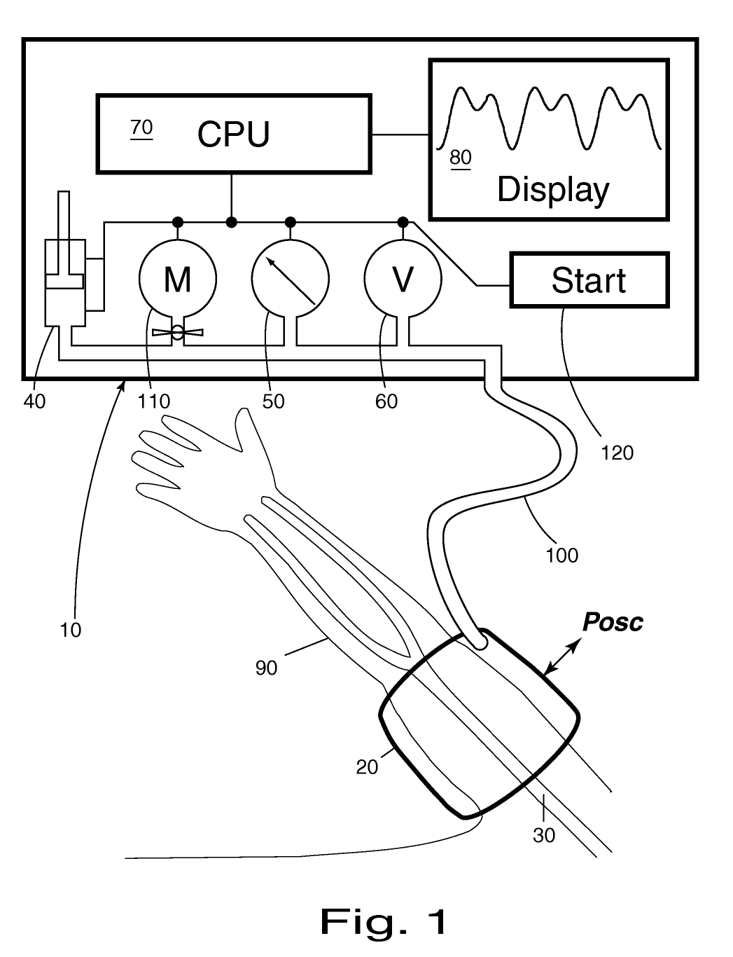

### Preferred Embodiment—FIG. 1

In one embodiment noninvasive blood pressure measurement apparatus 10 consists of the means 20 to variably compress the vessel 30, extrinsic oscillator 40 which introduces cyclical pressure perturbation (Posc)to the vascular bed 30, pressure sensor 50, which senses extrinsic vascular bed compression force (Pe), volume sensor 60, which senses vascular bed volume response to extrinsic cyclical perturbations, processing unit 70 and display unit 80.

In illustrated embodiment inflatable pressure cuff 20 is placed around the patients extremity 90 and is connected via one or more (preferably two) connecting hoses 100 to a measuring apparatus 10. Pressure cuff is connected to the pressure pump 110, oscillator 40, pressure sensor 50 and volume sensor 60. Processing unit 70 is connected to pressure sensor 50, volume sensor 60, pressure pump 110, oscillator 40, display 80 and user controls 120.

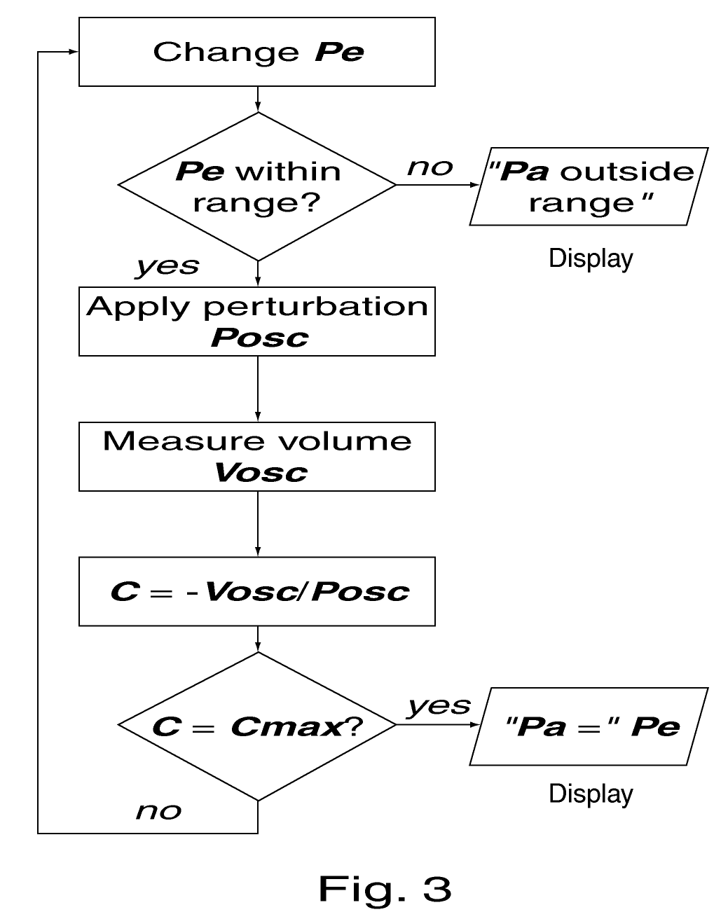

### Operation—FIGS. 3, 4, 6, 7

To measure the blood pressure Pa pneumatic pressure cuff 20 is fitted around the extremity and attached via the connecting hose 100 to the measuring unit 10. Pressure cuff 20 is inflated with the pressure pump 110. While pressure Pe is varied by the pressure pump 110, oscillator 40 ads extrinsic oscillatory component Posc. Pressure Pe is measured in the cuff 20 by the pressure sensor 50. Pressure sensor 50 reads average pressure (e.g. using low pass filter) and oscillatory pressure component Posc (e.g. high pass filter). Blood volume under the cuff V is measured with volume sensor 60. Oscillatory volume component is measured as Vosc using high pass filter or pressure and volume signal cross correlation. In another embodiment oscillator 40 is a sound wave generator and pressure sensor 50 is a microphone.

Cuff is inflated with the pump 110 and vessel compliance Cis calculated as C=Vosc/Posc.

Cuff is inflated to cover expected arterial pressure range.

While cuff pressure Pₑ is being changed, oscillatory pressure and volume components are measured and compliance C=−V_{osc}/P_{osc} is calculated.

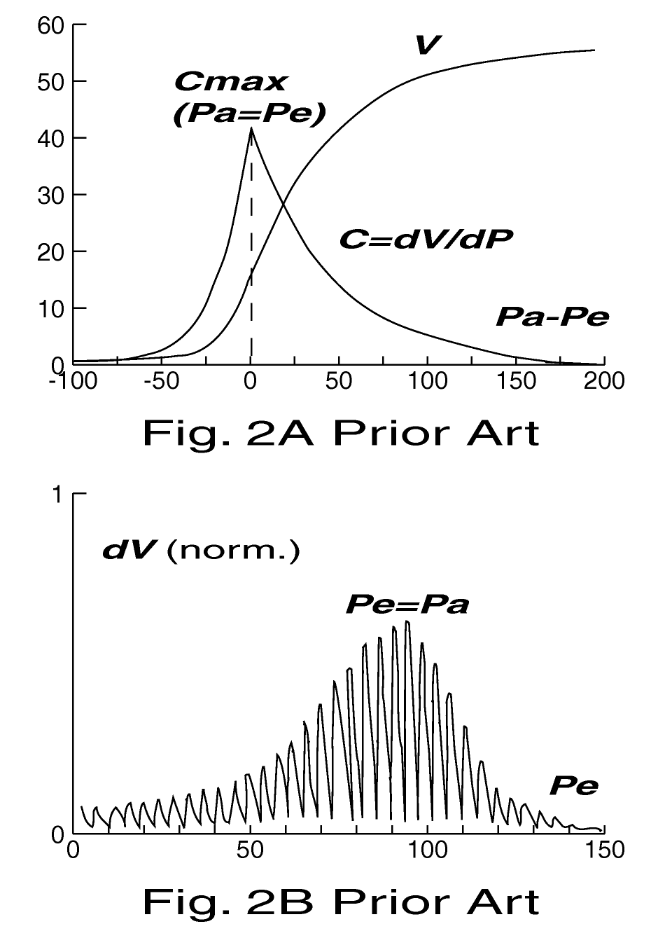

Vascular compliance C is maximal (C=Cmax) when the cuff pressure Pₑ approximates mean vascular pressure and transmural pressure=0 (FIG. 2A). When vascular bed is collapsed (Pₑ>>Pₐ), C becomes zero.

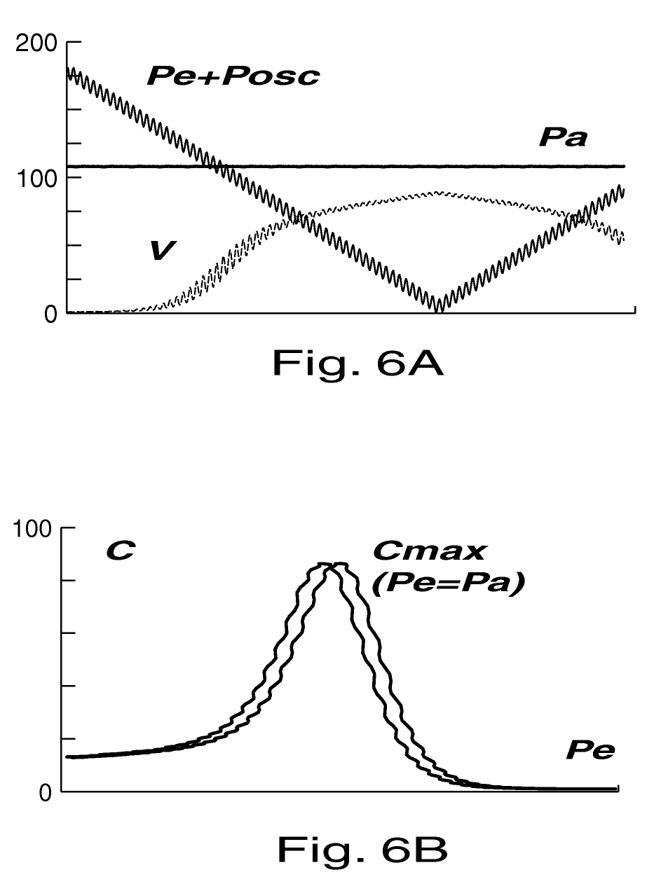

To assess vascular compliance C high fidelity measurements are taken over the range of Pₑ. C=Cmax when Pₑ=Pₐ (FIGS. 3, 6).

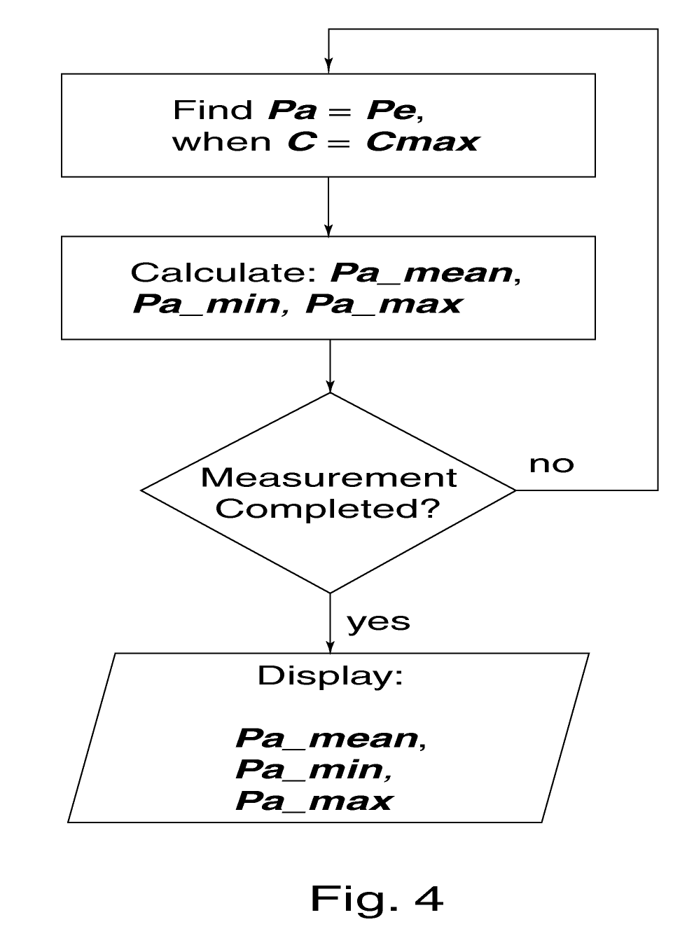

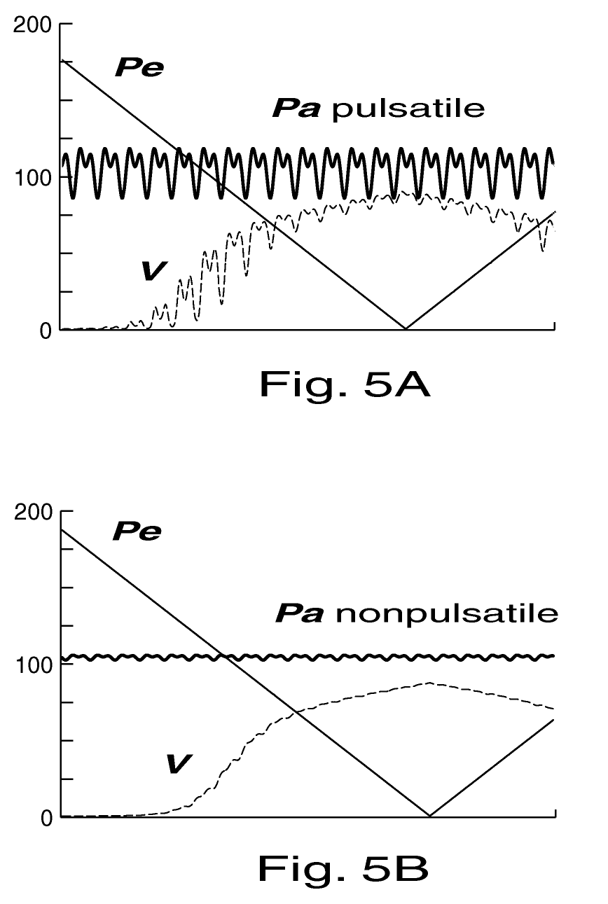

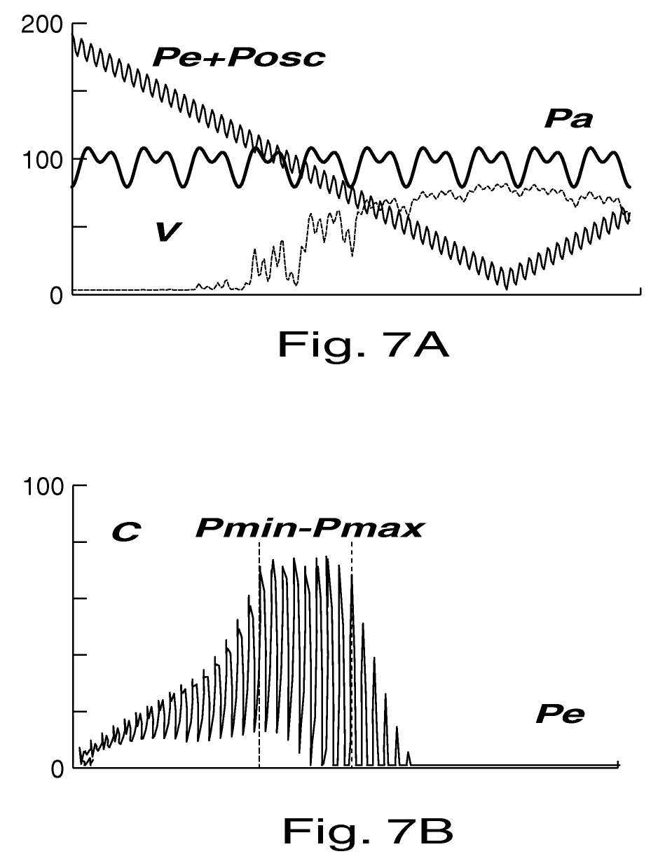

When arterial pressure is pulsatile or varies over time, plurality of compliance peaks C=Cmax at different external pressure Pe values are obtained. Cmax at highest external pressure Pe corresponds to high (systolic) and at lowest Pe corresponds to low (diastolic) arterial blood pressure (FIGS. 4, 7).

### Alternative Embodiments

Multiple alternative invention embodiments are possible depending on the vascular bed compression method 20, extrinsic perturbation mode 40 (vibration, acoustic wave, etc.), receiving volume sensor 60 modality and placement.

In an alternative embodiment vessel bed 30 or corresponding compartment can be compressed by cuff 20 which is filled with liquid to diminish cuff compliance.

In yet another embodiment, compression is performed applying direct pressure over the vessel with a tonometer. Using tonometry pressure is applied to the tissue covering the vessel or compartment rather than around the extremity. Tonometry is preferable way to measure intraocular pressure.

Tonometry (Prior art U.S. Pat. No. 4,269,193) allows to measure pressure in the specific artery/vein. Measuring pressure in two locations allow to evaluate pressure wave characteristics.

In yet alternative embodiments oscillator 40 utilizes electromechanical pneumatic, piezo, vibratory or acoustic perturbation.

In yet alternative embodiments oscillator 40 is located directly over the body part containing the vessel, combined with a vessel compression device 20 or over the body part distant from compression device 20.

In yet alternative embodiments volume sensor 60 senses changes in pressure in the cuff, volume in the cuff, Doppler signal (from blood or blood vessel wall), optical signal (e.g. scattering or border recognition), pletysmogram (photo, impedance, etc).

In yet alternative embodiments volume sensor 60 and pressure sensor 50 are close to the cuff or incorporated in the cuff 20. Closer placement of the oscillator/sensor diminishes lag for cuff compliance measurement and vascular compliance estimation.

In yet alternative embodiment extrinsic perturbation measuring unit is incorporated into standard NIBP measurement machine.

Commonly used NIBP machines are based on the oscillatory measurement method and changes Pe, while registering intrinsic oscillations. When Pe=Pa, oscillation amplitude reaches maximum (FIG. 2B). Attaching additional extrinsic oscillation measuring unit 10 to the NIBP hose/cuff connection allows incorporating extrinsic oscillations to assess vascular pressure. Pe is varied by the noninvasive machine; Posc is introduced, volume response Vosc is registered and compliance C=−Vosc/Posc is calculated. Compliance/pressure dependence is obtained C(Pe) in the measured range of Pe. Preferably external oscillations do not interfere with intrinsic oscillation registration (e.g. they are different frequency range).

F) Registering vascular wall motion in response to external oscillation with high frequency Doppler.

Same principles we described for measurement of the intravascular pressure apply to measure intraocular or any other compressible compartment pressure. Body compartments where pressure can be measured using extrinsic perturbation include but are not limited to venous, intraocular, bladder, intraabdominal, extremity.

### Advantages

From the description above a number of advantages of noninvasive pressure measurement become evident:
(1) Blood pressure can be measured in the absence of pulsatile flow (arrest, cardiac bypass, and cardiac assist).
(2) Blood pressure can be measures when blood pressure pulsation is very weak (shock, premature neonates).
(3) Blood pressure can be measured when blood pressure pulsation is irregular (arrhythmias) or changes rapidly.
(4) Blood pressure can be measured faster as it does not require extending the measurement over few cardiac cycles.
(5) Blood pressure can be measured at both low and high pressure values.
(6) Blood pressure can be measured in critically ill or trauma patients with hemodynamic instability.
(7) Method is automatic and does not require specialized training from the operator.
(8) Method avoids invasive arterial pressure monitoring for many patients and provides backup monitoring capability for others.
(9) Method allows estimation of CPR effectiveness during resuscitation.
(10) Method allows to measure pressure in the venous or other compressible nonpulsatile body compartments.

### Conclusions, Ramifications and Scope

Accordingly, described method using extrinsic perturbation allows measurement of blood pressure during critical situations when obtaining blood pressure is needed the most. It does not depend on intrinsic blood pressure oscillations, therefore can be applied to venous or any other compressible nonpulsatile body compartment or during arrhythmias. Method is devoid of limitations of current noninvasive pressure measurement methods as it can measure pressure even in the absence of regular arterial pressure oscillations.

It is noninvasive equivalent of having arterial line, but is simple to apply, does not require specialized invasive monitoring equipment, does not require qualified personnel to place and monitor invasive lines, does not have the risks of invasive lines. Method can be used in the hospital, ambulatory setting, patient's home or in the field.

Although description above contains many specificities, these should not be construed as limited the scope of the embodiment but as merely providing illustrations of some of the presently preferred embodiments. For example compartment where pressure can be measured using extrinsic perturbation is not limited to intravascular (arterial, venous), or ocular but also includes muscle or muscle group, liver, or any other compressible organ or compartment.

Thus the scope of the embodiments should be determined by the appended claims and their legal equivalents, rather than by the examples given.

### REFERENCES

Gedes L A. The direct and indirect measurement of blood pressure. Year Book Medical publishers, Chicago 1970, pp. 296.

What is claimed is:

1. A noninvasive device for measuring blood pressure in a body portion comprising:
(a) pressure application means for pressing the body portion, containing blood vessel;
(b) pressure changing means in said pressure application means for changing pressure level across a range which is expected to include blood pressure level;
(c) repetitive pressure perturbation means for superimposing pressure perturbation onto already established pressure level in said pressure application means;
(d) pressure sensing means in said pressure application means;
(e) vessel volume measurement means for measuring blood vessel volume under said pressure application means;
(f) compliance calculating means for calculating compliance as a ratio of the blood vessel volume change to the pressure perturbation at the each pressure level in said pressure application means; and
(g) a means of indicating blood pressure as the cuff pressure level, where said compliance is maximal.

2. The device for measuring blood pressure as recited in claim 1 wherein said pressure application means for pressing body portion containing blood vessel is inflatable pressure cuff.

3. The device for measuring blood pressure as recited in claim 2 wherein said inflatable pressure cuff for pressing body portion is filled with noncompliant fluid.

4. The device for measuring blood pressure as recited in claim 1 wherein said means of repetitive pressure perturbation are electromechanical.

5. The device for measuring blood pressure as recited in claim 1 wherein said means of repetitive pressure perturbation are pneumatic.

6. The device for measuring blood pressure as recited in claim 1 wherein said means of repetitive pressure perturbation are external vibratory.

7. The device for measuring blood pressure as recited in claim 1 wherein said vessel volume measurement means under said pressure application means are acoustic.

8. The device for measuring blood pressure as recited in claim 1 wherein said vessel volume measurement means under said pressure application means are plethysmographic.

9. The device for measuring blood pressure as recited in claim 1 wherein said vessel volume measurement means under said pressure application means are pressure measurement means in the cuff.

10. A noninvasive method to measure blood pressure in a body part which comprises the steps of:
(a) applying an external pressure to the body part comprising blood vessel;
(b) applying repetitive external pressure perturbation to the body part comprising blood vessel;
(c) registering oscillatory blood volume response of the body part where external pressure is applied;
(d) calculating compliance as a ratio of said volume response to said repetitive external pressure perturbation;
(e) changing the external pressure to obtain range of values expected to include intravascular pressure; then
(f) repeating steps b, c and d to obtain compliance for the each external pressure level; and
(g) displaying intravascular pressure level as the external pressure with maximal compliance.

11. The noninvasive method to measure blood pressure as recited in claim 10, further comprising:
(a) repeating steps a through g multiple times to obtain plurality of pressures where maximal compliance is achieved;
(b) selecting maximal, minimal and mean pressure out of the pressures plurality;
(c) displaying said maximal pressure as systolic blood pressure;
(d) displaying said minimal pressure as diastolic blood pressure; and
(e) displaying said mean pressure as mean blood pressure.

12. The noninvasive method to measure blood pressure as recited in claim 10 wherein the step of applying repetitive external pressure perturbation to the body part comprising blood vessel is performed by providing external vibration.

13. The noninvasive method to measure blood pressure as recited in claim 10 wherein the step of applying repetitive external pressure perturbation to the body part comprising blood vessel is performed by providing electromechanical oscillation.

14. The noninvasive method to measure blood pressure as recited in claim 10 wherein the step of applying repetitive external pressure perturbation to the body part comprising blood vessel is performed by providing pneumatic oscillation.

15. The noninvasive method to measure blood pressure as recited in claim 10 wherein the step of registering oscillatory blood volume response of the body part under said cuff by registering the oscillatory volume ultrasonically.

16. The noninvasive method to measure blood pressure as recited in claim 10 wherein the step of registering oscillatory blood volume response of the body part under said cuff by registering the oscillatory volume plethysmografically.

17. A noninvasive device for measuring pressure in a body compartment comprising:
(a) pressure application means for pressing the body portion, containing compartment;
(b) pressure changing means in said pressure application means for changing pressure level across a range which is expected to include compartment pressure level;
(c) repetitive pressure perturbation means for superimposing pressure perturbation onto already established pressure level in said pressure application means;
(d) pressure sensing means in said pressure application means;
(e) compartment volume measurement means for measuring compartment volume under said pressure application means;
(f) compliance calculating means for calculating compliance as a ratio of the compartment volume change to the pressure perturbation at the each pressure level in said pressure application means; and
(g) a means of indicating compartment pressure as the external pressure level, where said compliance is maximal.

18. The device for measuring pressure in a body compartment as recited in claim 17 wherein said pressure application means for pressing body portion contain fluid.

19. In a method of noninvasive oscillatory blood pressure measurement of the type wherein external pressure is applied to the body part comprising blood vessel and compression oscillation from blood pressure pulsations is registered across the range of compression and maximal oscillation coincides with external pressure and blood pressure equilibration wherein the improvement comprises:
(a) applying repetitive external pressure perturbation to the body part comprising blood vessel;
(b) registering oscillatory blood volume response of the body part where external pressure is applied;
(c) calculating compliance as a ratio of said volume response to said repetitive external pressure perturbation for the range of external pressure values; and
(d) displaying intravascular pressure level as the external pressure with maximal compliance whereby blood pressure can be measured in the absence of pulsatile blood flow or when intrinsic pressure oscillations are diminished.
